(19) United States
(12) Patent Application Publication
(10) Pub. No.: US 2024/0260903 A1
(43) Pub. Date: Aug. 8, 2024

(54) SYSTEM AND METHOD FOR DETECTING AND REMOVING PERIODIC NON-PHYSIOLOGICAL ARTIFACT FROM EVOKED POTENTIALS

(71) Applicant: SafeOp Surgical, Inc., Hunt Valley, MD (US)

(72) Inventors: Cameron Snow, Phoenix, MD (US); Richard O'Brien, Hunt Valley, MD (US); Gregg Johns, Toronto (CA)

(21) Appl. No.: 18/641,240

(22) Filed: Apr. 19, 2024

Related U.S. Application Data
(62) Division of application No. 15/710,749, filed on Sep. 20, 2017, now Pat. No. 11,986,321.
(60) Provisional application No. 62/398,419, filed on Sep. 22, 2016.

(51) Int. Cl. A61B 5/00; A61B 5/0205; A61B 5/316; A61B 5/377; A61N 1/18; A61N 1/36; G11B 20/24; G16H 40/63

(52) U.S. Cl. CPC A61B 5/7203; A61B 5/0205; A61B 5/316; A61B 5/377; A61B 5/7217; A61N 1/36014; G11B 20/24; G16H 40/63; A61B 2560/0247; A61N 1/18

(57) ABSTRACT

An automated electrophysiological response analysis apparatus for identifying and eliminating signals having non-physiological artifact noise from an averaged evoked potential signal, wherein the apparatus is adapted to identify in an electrophysiological response at least one characteristic representative of non-physiological artifact noise to classify the signal as an artifact signal and remove the artifact signal from a collection of signals used to generate the averaged evoked potential signal.

### CROSS REFERENCE TO RELATED MATTER

This is a divisional of U.S. patent application Ser. No. 15/710,749, filed on Sep. 20, 2017 and titled SYSTEM AND METHOD FOR DETECTING AND REMOVING PERIODIC NON-PHYSIOLOGICAL ARTIFACT FROM EVOKED POTENTIALS (“the '749 Application”) which claims priority to U.S. Provisional Application No. 62/398,419, filed Sep. 22, 2016 and titled SYSTEM. METHOD NAD COMPUTER ALGORITHM FOR DETECTING AND REMOVING PERIODIC NON-PHYSIOLOGICAL ARTIFACT FROM EVOKED POTENTIALS (“the '419 Provisional Application”). The entire disclosure of the '749 Application and the '419 Provisional Application are hereby incorporated herein.

### BACKGROUND

The present invention relates generally to the field of removing noise from recorded waveform signals, such as evoked potentials (EPs), and more particularly to a system, method, and computer algorithm for automatically detecting periodic non-physiological artifact and removing if from the EPs.

Bio-electrical potentials such as somatosensory evoked potentials (ssep) are summated electrical potentials usually recorded after stimulating a peripheral nerve or parts of the nervous system. Monitoring patients by recording waveforms such as somatosensory evoked potentials during surgery has been shown to allow early identification of impending nerve injury. The difficulty with analyzing and classifying the waveforms lies in the wide variation in the amplitude, frequency and shape of the waveforms. These variations are caused by many factors including anesthesia and any preexisting abnormalities of the nerves, however one primary cause is electrical interference from ambient electrical noise or from other devices, such as pacemakers.

External pacemakers are an essential part to some cardiac surgeries. Generally, a transcutaneous pacing system is introduced when the patient starts to experience bradycardia (abnormally slow heart action), or if they develop any other irregularities in their heartbeat. Some patients may have internal pacemakers already in place for similar issues. Unfortunately, the electric signals produced by pacing systems are large relative to the ssep being recorded, but often smaller than can be reliably captured by a voltage threshold rejection classifier and contain frequencies not typically excluded by traditional high-pass and low-pass filters used to condition the ssep signals. The electrical interference from the pacing system therefore can represent significant power in the recorded waveforms.

Embodiments herein generally relate to improved systems and methods to automatically and more fully remove pacemaker or similar artifact from recorded ssep signals. The systems and methods overcome many drawbacks caused by non-physiologic, such as pacemaker, noise and drawbacks of current attempts to eliminate such noise. For example, described herein according to some embodiments are systems, methods, and computer signal processing algorithms for the removal of these types of periodic artifacts from evoked potential signals, which can be performed quickly and in real-time without significant processing overhead and without significantly altering the characteristics of the signal of interest.

### SUMMARY OF THE INVENTION

One embodiment of the invention relates to an automated EP analysis apparatus for identifying and eliminating signals having non-physiological artifact noise from an averaged EP signal, wherein the apparatus is adapted to identify in an electrophysiological response at least one characteristic representative of non-physiological artifact noise to classify the signal as an artifact signal and remove the artifact signal from a collection of signals used to generate the averaged EP signal.

In some embodiments, the non-physiological artifact noise is pacing artifact from a pacemaker. In some embodiments, the at least one characteristic is at least one from the group consisting of: a minimum slope, minimum amplitude, a maximum rise time, a maximum fall time, a minimum peak duration, and a maximum activity between a rising edge and a falling edge of the signal.

In some embodiments, the apparatus further comprises: an output operable to couple to at least one stimulating electrode to stimulate one or more peripheral nerves of a patient, an input operable to couple to at least one recording electrode to record the electrophysiological response comprising the resultant electrical waveforms generated by a nervous system in response to the one or more stimulating electrodes, and at least one processor, coupled to said output and said input, operable to cause the apparatus to identify in the electrophysiological response at least one characteristic representative of non-physiological artifact noise to classify the signal as an artifact signal and remove the artifact signal from a collection of signals used to generate the averaged EP signal.

Another embodiment of the invention relates to a method for identifying and eliminating signals having non-physiological artifact noise from an averaged EP signal. The method includes recording resultant waveforms generated by a nervous system, resulting from stimulation of one or more nerves of the nervous system, identifying in the resultant waveforms at least one characteristic representative of non-physiological artifact noise, if the at least one characteristic is present in a resultant waveform, classifying the waveform as an artifact signal, and generating an averaged EP signal using only the resultant waveforms that have not been classified as an artifact signal.

In some embodiments, generating an averaged EP signal using only the resultant waveforms that have not been classified as an artifact signal is achieved by removing the artifact signal from a collection of signals used to generate the averaged EP signal. In some embodiments, the non-physiological artifact noise is pacing artifact from a pacemaker. In some embodiments, the at least one characteristic is at least one from the group consisting of: a minimum slope, minimum amplitude, a maximum rise time, a maximum fall time, a minimum peak duration, and a maximum activity between a rising edge and a falling edge of the signal.

In some embodiments, the method further includes identifying a plurality of characteristics representative of non-physiological artifact noise by analyzing the signal for a first characteristic and if the first characteristic is present, continuing to analyze the signal for a second characteristic.

Another embodiment of the invention relates to an automated Evoked Potential (EP) analysis algorithm for monitoring, detecting, identifying and eliminating non-physiological artifact noise in the physiological system from which the EPs or Ensemble Averages (EA) are obtained, wherein the algorithm identifies artifacts in the individual electrophysiological responses (ER) to repetitive stimulation allowing rejection of those containing such artifacts from the ensemble average.

In some embodiments, the non-physiological artifact noise is noise from a pacemaker.

In some embodiments, there is an apparatus for monitoring, detecting, identifying and eliminating non-physiological artifact noise in the physiological system from which the EPs or Ensemble Averages (EA) are obtained, that implements the algorithm described above.

In some embodiments, the apparatus further comprises a system that processes ER sequentially looking for a series of characteristics. In some embodiments, the apparatus further comprises a system that processes ER sequentially looking for a single primary characteristic. In some embodiments, the apparatus further comprises a system that iteratively processes ER meeting a preceding identified characteristic for a further characteristic and stops processing if a characteristic is not found, and rejects a ER from an ensemble average if all characteristics are found.

In some embodiments, the apparatus further comprises a system in which the characteristics comprise but are not limited to amplitude, rise time, fall time, peak duration and pre and post peak slope. In some embodiments, the apparatus further comprises a system in which the characteristics can be altered. In some embodiments, the apparatus further comprises a system in which integrate such apparatus into other devices in a surgical environment. In some embodiments, the apparatus can feed information to other devices in the surgical environment that allows these devices to identify the presence of pacer activity. In some embodiments, the apparatus can obtain information from an anesthesia or blood pressure machine or pacemaker to calculate when changes in EP waveforms are due to anesthesia or physiologic or non-physiologic changes.

### BRIEF DESCRIPTION OF THE DRAWINGS

FIG. 1A depicts several evoked potential signals before a pacing signal was applied.

FIG. 1B depicts the evoked potential signals after a pacing signal was applied, showing the effect of the pacing signal on the recorded waveforms.

FIG. 2A depicts an averaged signal compared with a baseline signal prior to a pacing signal being applied.

FIG. 2B depicts an averaged signal compared with the baseline when an external pacing signal being applied.

FIG. 3 shows a collection of approximately 1000 ERs obtained during a surgery, to depict the characteristics of a pacing pulse signal relative to the ER signals obtained by the system.

FIG. 4 shows five individual ERs, where one of the signals (second from top) is contaminated with a pacing artifact.

FIG. 5 is a table of threshold values for identifying characteristics of an artifact signal, according to one embodiment.

FIG. 6A depicts an averaged somatosensory evoked potential recording contaminated by pacemaker artifact.

FIG. 6B depicts the somatosensory evoked potential recording of FIG. 6A, but with the signal recordings containing pacemaker artifact rejected before averaging.

FIG. 7 depicts an exemplary diagram of the system according to an exemplary embodiment of the present invention.

FIG. 8 depicts an exemplary embodiment of a computer system that may be used in association with an exemplary embodiment of the present invention.

### DETAILED DESCRIPTION

Bio-electrical potentials such as somatosensory evoked potentials (ssep) are summated electrical potentials usually recorded after repeatedly stimulating a peripheral nerve or parts of the nervous system. Monitoring patients by recording waveforms such as somatosensory evoked potentials during surgery has been shown to allow early identification of impending nerve injury. Such monitoring is performed with sophisticated, multichannel amplifier and display equipment. This monitoring is often fraught with difficulties due to the small size of potentials and large amounts of ongoing noise which makes recognizing significant changes and when to alert for these changes difficult.

The difficulty with analyzing and classifying the waveforms lies in the wide variation in the amplitude, frequency and shape of the waveforms. These variations are caused by many factors including anesthesia and any preexisting abnormalities of the nerves, however the main cause is electrical interference from ambient electrical noise or from other devices such as pacemakers.

External pacemakers are an essential part to some cardiac surgeries. Generally, a transcutaneous pacing system is introduced when the patient starts to experience bradycardia (abnormally slow heart action), or if they develop any other irregularities in their heartbeat. The anesthesiologist may first attempt to remedy the patient with a drug, but if that fails, pacing is introduced in order to regulate the heartbeat. By providing timed pulses while also recording the hearts activity, the pacing system can bring the patient's heart back into a consistent and natural rhythm. Without the external pacing system, the patient would be more likely to go into cardiac arrest. Other patients may have internal pacemakers in place for similar issues.

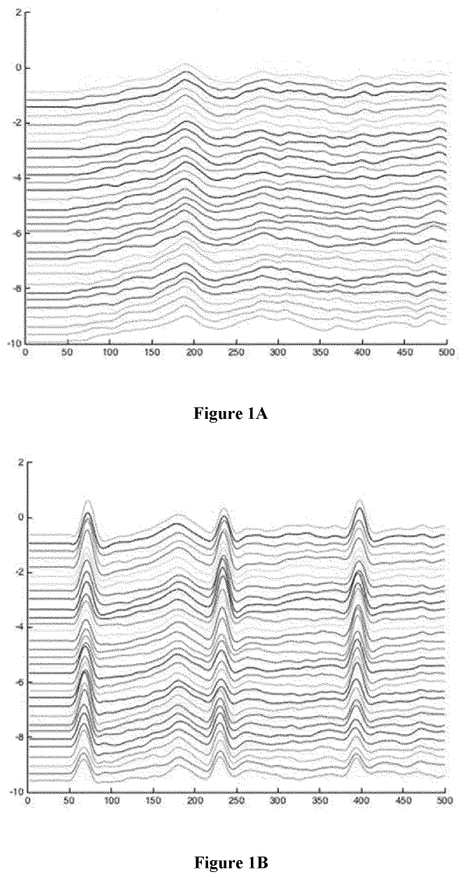

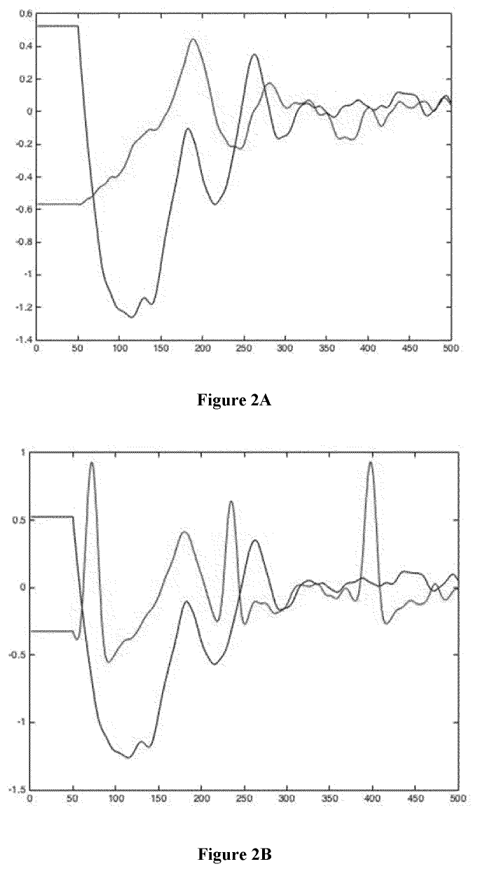

Unfortunately, the electric signals produced by pacing systems are large relative to the somatosensory evoked potential signals being recorded, but often smaller than can be reliably captured by a fixed threshold rejection classifier and contain frequencies not typically attenuated by high- and low-pass filters used to condition the sseps. These electrical signals from the pacing system therefore end up being a major source of noise in the recorded waveforms, as shown in FIGS. 1 and 2. FIG. 1A depicts several signals before a pacing signal was applied, and FIG. 1B depicts the obtained signals after a pacing signal was applied, showing the effect of the pacing signal on the recorded waveforms. Similarly, FIG. 2A depicts an averaged signal compared with a baseline signal prior to a pacing signal being applied, and FIG. 2B depicts an averaged signal compared with the baseline with an external pacing signal being applied. It can be observed how an external pacing signal effects the physiological signals obtained.

Although electronic and digital filters are employed to attenuate noise from the signals and allow better viewing and interpretation of the waveforms, these filters typically filter in two ways, by limiting recoded waveform frequency range or rejecting signals of high amplitude that contain clear artifact. waveform frequency or alteration of the overall data such that only partial removal of noise occurs or the morphology, amplitude or even presence/absence of the underlying potentials that he user is interested in are changed making interpretation difficult and highly dependent on experience. This in turn may lead to erroneous interpretations.

Standard high- and low-pass filters that limit the bandwidth of the recordings, or classifiers that reject raw recordings over a certain amplitude threshold (rejection threshold classifier) may have difficulty removing pacemaker or pacemaker like artifacts from evoked potential recordings, leading to inability to record accurate signals.

There are described methods for detecting and removing pacemaker artifact from electroencephalographic signals, however these primarily apply to continuous EEG or ECG recordings and not to stimulus evoked monitoring. These methods are also complex and introduce significant processor overhead.

U.S. Pat. No. 8,440,903 (Donehoo) describes a variable amplitude threshold that identifies and eliminates a pacemaker's pulse from data. Once the initial threshold is surpassed by a pacing artifact, the threshold decays in magnitude so that any of the characteristic “ringing” is cut out. However, evoked potentials are not affected by the ringing created by the pacing pulses, and therefore do not have a major need for a variable threshold classifier which adds complexity and additional processing.

Accordingly, certain embodiments disclosed herein provide a lean and fast computer algorithm running on software installed on an evoked potential monitoring machine, which can be used in any surgery or situation where a patient is at risk, to detect and remove confounding pacemaker or similar noise and display the underlying signals more accurately to the user(s).

In an exemplary embodiment of the present invention a system, method, and computer algorithm for identification and removal of pacemaker and pacemaker-like artifact from electrophysiological EPs is disclosed. In this application, an EP is defined as a voltage versus time signal obtained by ensemble averaging (EA) the electrophysiological responses (ER) to repetitive stimulation of a specific sensory neural system detected using suitable electrodes. Examples of EPs are somatosensory, auditory or visual EPs. Ensemble averaging of the electrophysiological responses may be performed according to the system and methods described in U.S. Patent Publication 2014/0020178 entitled “System, Method, and Computer Algorithm and Characterization and Classification of Electrophysiological Evoked Potentials,” which is herein incorporated by reference in its entirety.

The methods and algorithms are applied to every ER signal recoded after each stimulation that survives any initial frequency and amplitude rejection filtering, and prior to summation of those timed signals into a EA The algorithm establishes the presence or absence of a pacemaker or similar artifact in each ER, and then excludes those in which it is present from the EA.

In an exemplary embodiment of the present invention, in order to perform this task with higher efficiency, the algorithm processes the individual signals sequentially in a gated fashion looking for the presence or absence of several characteristics and only performing further examination for remaining characteristics if the preceding characteristic is present. If all characteristics are present, indicative of noise from a pacing or other similar pulse, it rejects the ER from the EA.

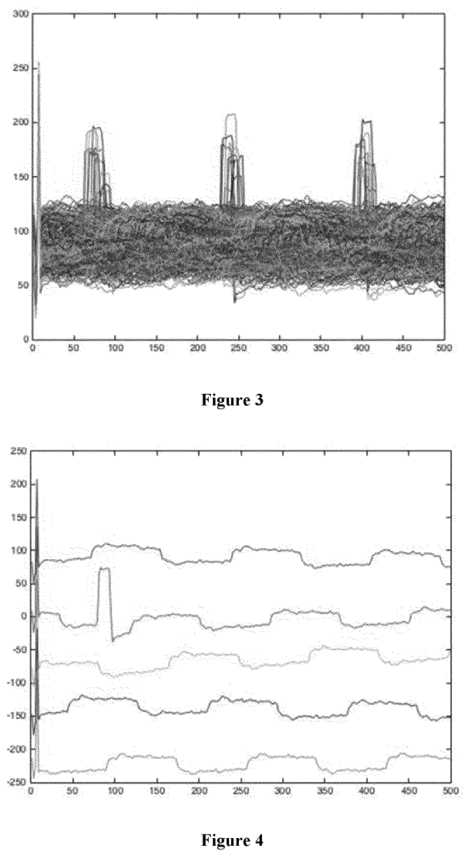

FIG. 3 shows a collection of approximately 1000 ERs obtained during a surgery, to depict the characteristics of a pacing pulse signal relative to the ER signals obtained by the system. FIG. 4 shows five individual ERs, where one of the signals (second from top) is contaminated with a pacing artifact. As shown in FIGS. 3 and 4, the pacing pulse has several characteristics that distinguish it from the ER signals. Accordingly, several key characteristics of the pacing artifact are focused upon in order to identify contaminated traces. Since the pacing artifact resembles a square wave, the characteristics identified as important are the magnitude of the rising and falling edges, the amplitude, and the level of activity in between the edges. The order in which the algorithm examines these features is decided by the degree of feature's definition. The most well-defined traits of the artifact are the distinctive rising and falling edges, therefore the algorithm may primarily look for a steep positive slope followed by a steep negative slope within some time range. The amplitude is then considered the next best defined trait, since the pacing artifact is significantly greater in magnitude than the rest of the data. Then, the level of activity between the two edges may be investigated.

Accordingly, in an exemplary embodiment of the present invention, the algorithm first looks for any ER that contain sudden increases in amplitude (signal having a steep rising edge), characteristic of non-physiological artifact. If none is present, no further processing is required. If this characteristic is present, the algorithm looks for the presence of a sudden decrease in amplitude occurring within a specified time or window length from initial increase. If this characteristic is present, the algorithm continues to analyze each ER through a series of characteristics which may include but are not limited to amplitude, rise time, peak duration and pre and post peak slope. If all defined characteristics are present it rejects the signal from the ensemble averaging. This process is described in greater detail below.

Since the artifact has a steep rising edge followed by a steep falling edge, the slopes of the original data are firstly taken into consideration. After taking the first difference of the pre-collected raw data, the algorithm would use a threshold trigger to start the identification process. Following an initial large positive slope, a pre-defined time window is set, starting at the point of threshold triggering, so that if there is a large negative slope within the window, the data has positively identified a first characteristic of a pacing signal (or similar noise), and the identification process moves on. If there is not a large negative slope within that time window, the algorithm ignores the original window starting point, and continues looking through that trace for a different positive slope.

If a large negative slope was confirmed within the time window, the amplitude of this deviation in the data is calculated. The amplitude is calculated by first recognizing a base for the data. The artifacts' onset and trough are not often the same voltage, and should be averaged to achieve a more consistent amplitude check. Once the base is established, the difference between the voltage of the base and the midpoint of the artifact is considered to be the amplitude. If the amplitude of the deviation is large enough, the algorithm continues the identification process, and if the amplitude of the deviation is smaller than the threshold value, then the process is stopped and the algorithm continues to look for the next large positive slope. This step is necessary to confirm that the deviation is large enough to be considered a pacing artifact, as there is often oscillatory noise in the raw data that contains relatively large positive and negative slopes in a small window.

Finally, the slope between the edges is examined to confirm if there is consistent data with little slope between the edges. The pacing artifact resembles a square wave, and therefore has little activity in between the two edges. If there is too much activity between the edges, this suggests that the deviation being analyzed is noise and not artifact. If the data between the two edges is “flat” enough, then the trace is identified as having an artifact, and the algorithm stops looking for a large positive slope. To be considered flat the data between the two edges has to have several data points with little to no slope.

In order to detect the characteristics described above to identify the traces with pacing artifact, threshold values for each piece of data is set. In some embodiments, the threshold values are set as shown in FIG. 5. A slope threshold (to identify a steep rising and falling edge) describes a minimum rate of change required to alert the algorithm to the possible existence of pacing artifact. In some embodiments, the slope minimum is 15. A window length describes the window of time in which the algorithm looks for the large positive and negative slope (i.e., the steep rising and falling edge). In some embodiments, the window length is 2.0 ms. In other embodiments the window length may be between 2.0 to 2.5 ms. An amplitude threshold describes the minimum amplitude of the artifact, to confirm that processing to determine presence of a pacing artifact should continue on the current signal. In some embodiments, the amplitude minimum is 30 μV. Finally, the activity threshold describes the minimum number of data points to compare, to determine variability and slope between data points. In some embodiments, the minimum number of data points between the rising and falling edges, to determine activity, is three.

If the algorithm studies the entire trace as described above, and an artifact has not been identified, then the trace is identified as being “clean”. The clean traces are then separated into one data set, while the “dirty” traces (those were a pacing artifact is identified) are separated into another. The clean data set can then be used to make an EA that would be void of pacing artifact. Since pacemaker artifact occurs out of phase with typical evoked potential stimuli and usually at a much slower rate, only a small subset of raw time recordings shows the artifact as present and exclusion of those from the ensemble average does not affect the overall integrity of the ensemble average (EA).

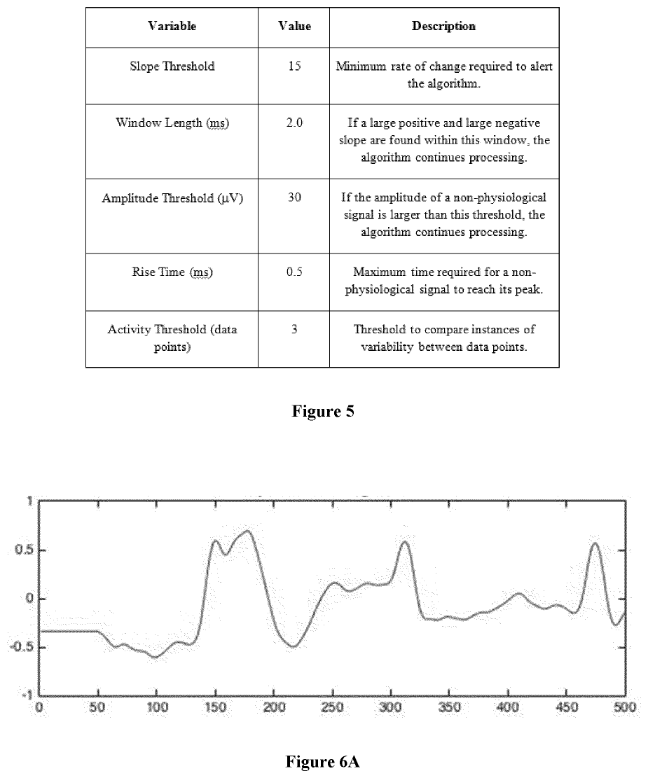

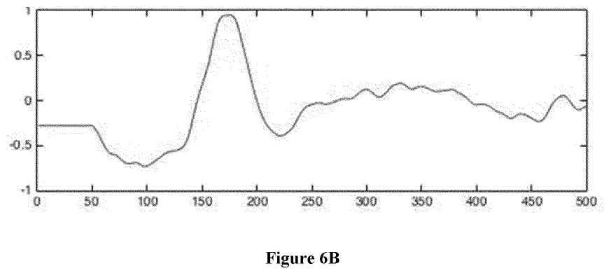

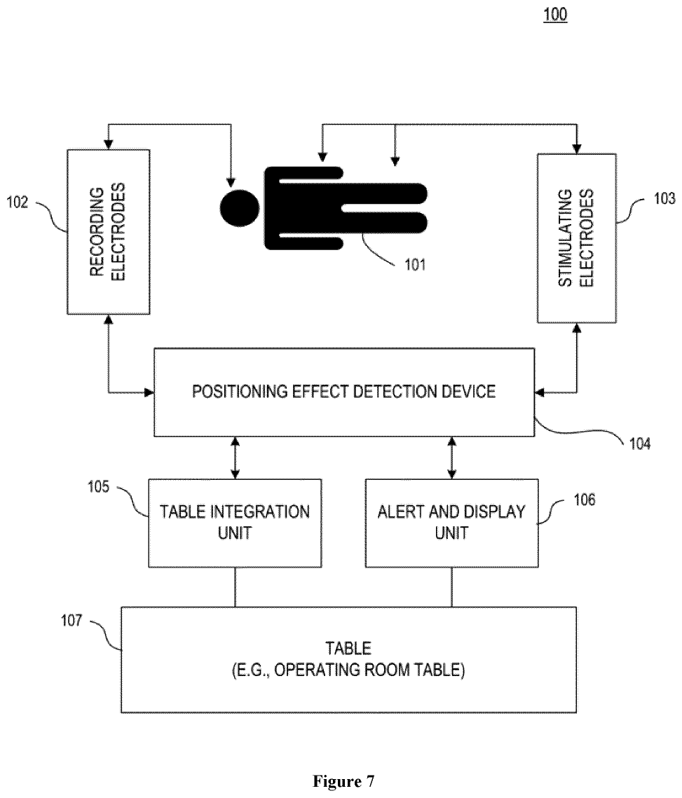

FIGS. 6A and 6B provide a comparison showing the effect of the above-described methods on the obtained EA FIG. 6A depicts an averaged somatosensory evoked potential recording contaminated by pacemaker artifact, while FIG. 6B depicts the somatosensory evoked potential recording of FIG. 6A, but with the signal recordings containing pacemaker artifact rejected before averaging. Accordingly, the EA of FIG. 6B provides a more accurate depiction of the signals being obtained by the system, in order to make a more accurate analysis of signals and determination of whether a nerve injury is impending.

FIG. 7 depicts an exemplary diagram of a system according to an exemplary embodiment of the present invention. According to an exemplary embodiment, the system 100, which may be coupled to a patient 101, may include, e.g., but not limited to, one or more recording electrodes 102, one or more stimulating electrodes 103, a positioning effect detection device (PEDD) 104, a table integration unit 105, an alert and display (or other output) unit 106, and a table 107. In an exemplary embodiment, the table 107 may include, e.g., but not limited to, any surface upon which the patient 101 may be placed, a bed, a chair, an operating room table, a pre-op table, and/or a post-op table, etc. Related or additional functions, components, capabilities, details, and other characteristics of such a system, not described herein, are described in U.S. Pat. No. 9,211,074, entitled “System, Method, Apparatus, Device, and Computer Program Product for Automatically Detecting Positioning Effect,” which is hereby incorporated by reference in its entirety.

According to an exemplary embodiment, the recording electrodes 102 may be coupled to the head, neck, spine, arms, legs, trunk, Erb's point and/or torso of the patient 101, and stimulating electrodes 103 may be coupled to the arms and/or legs of the patient 101.

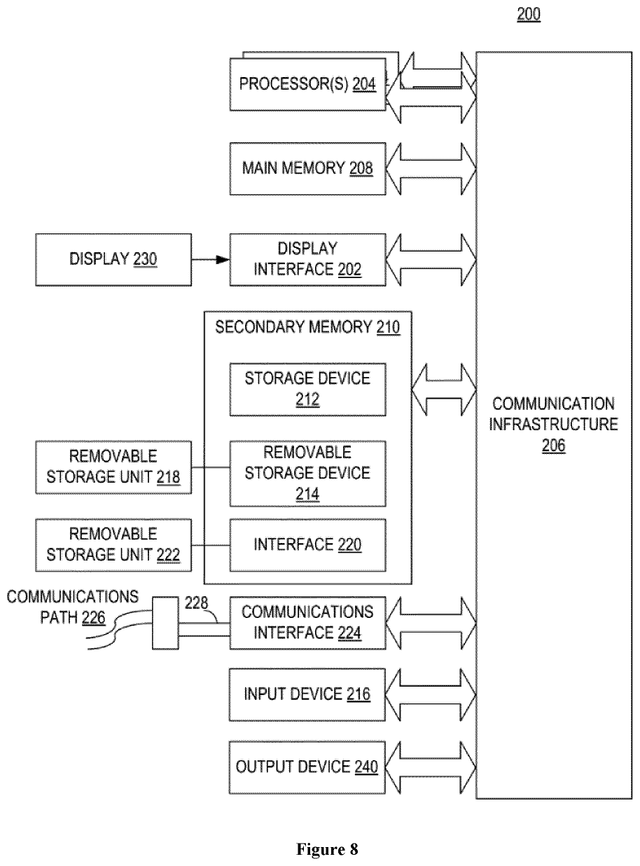

According to an exemplary embodiment, the PEDD 104 may be electronically coupled to recording electrodes 102 and stimulating electrodes 103. In an exemplary embodiment, the PEDD 104 may be part of, may be coupled to, and/or may include, a computer. According to an exemplary embodiment, the PEDD 104 may include a computer, such as, e.g., but not limited to, the computer set forth in and described further below with reference to FIG. 8. In an exemplary embodiment, PEDD 104 may be electrically, electronically, and/or mechanically coupled to the table integration unit 105 and/or the alert and display unit 106.

In an exemplary embodiment, the PEDD 104 may detect positioning effect in a patient 101 lying on the table 107 using the stimulating electrodes 103 and the recording electrodes 102. According to an exemplary embodiment, the PEDD 104 may communicate positioning effect information using the alert and display unit 106 to, e.g., operating room personnel.

According to an exemplary embodiment, the PEDD 104 may stimulate sensory or mixed nerves of the patient using the stimulating electrodes 103 to produce EPs. In an exemplary embodiment, a PEDD 104 may be attached, coupled and/or connected to the patient 101 with stimulating electrodes 103, e.g., near the arms or legs over peripheral nervous structures, such as, but not limited to, e.g., the ulnar nerves, median nerves and posterior tibial nerves.

According to an exemplary embodiment, the PEDD 104 may use the recording electrodes 102 to detect ERs generated by a patient's nervous system in response to the stimulation from the stimulating electrodes 103. These ERs may then be subject to the analysis described above to identify a pacing artifact and remove the signal containing the artifact from the averaged EPs.

According to an exemplary embodiment, based on the observed EPs, the PEDD 104 may identify potential positioning effect injuries caused by positioning of the patient 101. In an exemplary embodiment, the PEDD 104 may detect changes in the EPs, such as, e.g., but not limited to, changes in latency, changes in amplitude or changes in morphology. According to an exemplary embodiment, changes, such as, e.g., but not limited to, reductions or aberrations in the EPs may indicate a positioning effect. In an exemplary embodiment, the PEDD 104 may identify a particular nerve structure affected by positioning effect based on the EPs. The PEDD 104 may further recommend actions to ameliorate the positioning effect by recommending changes in position. In one exemplary embodiment, the PEDD 104 may move the patient automatically so as to prevent positioning effect injury to the patient 101.

FIG. 8 depicts an exemplary embodiment of a computer system 200 that may be used in association with, in connection with, and/or in place of, e.g., but not limited to, any of the foregoing components and/or systems.

The present embodiments (or any part(s) or function(s) thereof) may be implemented using hardware, software, firmware, or a combination thereof and may be implemented in one or more computer systems or other processing systems. In fact, in one exemplary embodiment, the invention may be directed toward one or more computer systems capable of carrying out the functionality described herein. An example of a computer system 200 is shown in FIG. 8, depicting an exemplary embodiment of a block diagram of an exemplary computer system useful for implementing the present invention. Specifically, FIG. 8 illustrates an example computer 200, which in an exemplary embodiment may be, e.g., (but not limited to) a personal computer (PC) system running an operating system such as, e.g., (but not limited to) WINDOWS MOBILE™ for POCKET PC, or MICROSOFT® WINDOWS® NT/98/2000/XP/CE/7/VISTA, etc. available from MICROSOFT® Corporation of Redmond, Wash., U.S.A., SOLARIS® from SUN® Microsystems of Santa Clara, Calif, U.S.A., OS/2 from IBM® Corporation of Armonk, N.Y., U.S.A., Mac/OS from APPLE® Corporation of Cupertino, Calif, U.S.A., etc., or any of various versions of UNIX® (a trademark of the Open Group of San Francisco, Calif, USA) including, e.g., LINUX®, HPUX®, IBM AIX®, and SCO/UNIX®, etc. However, the invention may not be limited to these platforms. Instead, the invention may be implemented on any appropriate computer system running any appropriate operating system. In one exemplary embodiment, the present invention may be implemented on a computer system operating as discussed herein. An exemplary computer system, computer 200 is shown in FIG. 8. Other components of the invention, such as, e.g., (but not limited to) a computing device, a communications device, a telephone, a personal digital assistant (PDA), a personal computer (PC), a handheld PC, client workstations, thin clients, thick clients, proxy servers, network communication servers, remote access devices, client computers, server computers, routers, web servers, data, media, audio, video, telephony or streaming technology servers, etc., may also be implemented using a computer such as that shown in FIG. 8.

The computer system 200 may include one or more processors, such as, e.g., but not limited to, processor(s) 204. The processor(s) 204 may be connected to a communication infrastructure 206 (e.g., but not limited to, a communications bus, cross-over bar, or network, etc.). Various exemplary software embodiments may be described in terms of this exemplary computer system. After reading this description, it will become apparent to a person skilled in the relevant art(s) how to implement the invention using other computer systems and/or architectures.

Computer system 200 may include a display interface 202 that may forward, e.g., but not limited to, graphics, text, and other data, etc., from the communication infrastructure 206 (or from a frame buffer, etc., not shown) for display on the display unit 230.

The computer system 200 may also include, e.g., but may not be limited to, a main memory 208, random access memory (RAM), and a secondary memory 210, etc. The secondary memory 210 may include, for example, (but may not be limited to) a hard disk drive 212 and/or a removable storage drive 214, representing a floppy diskette drive, a magnetic tape drive, an optical disk drive, a magneto-optical disk drive, a compact disk drive CD-ROM, a digital versatile disk (DVD), a write once read many (WORM) device, a flash memory device, etc. The removable storage drive 214 may, e.g., but not limited to, read from and/or write to a removable storage unit 218 in a well-known manner. Removable storage unit 218, also called a program storage device or a computer program product, may represent, e.g., but not limited to, a floppy disk, a magnetic tape, an optical disk, a magneto-optical disk, a compact disk, a flash memory device, etc. which may be read from and written to by removable storage drive 214. As will be appreciated, the removable storage unit 218 may include a computer usable storage medium having stored therein computer software and/or data.

In alternative exemplary embodiments, secondary memory 210 may include other similar devices for allowing computer programs or other instructions to be loaded into computer system 200. Such devices may include, for example, a removable storage unit 222 and an interface 220. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units 222 and interfaces 220, which may allow software and data to be transferred from the removable storage unit 222 to computer system 200.

Computer 200 may also include an input device 216 such as, e.g., (but not limited to) a mouse or other pointing device such as a digitizer, and a keyboard or other data entry device (none of which are labeled).

Computer 200 may also include output devices 240, such as, e.g., (but not limited to) display 230, and display interface 202. Computer 200 may include input/output (I/O) devices such as, e.g., (but not limited to) communications interface 224, cable 228 and communications path 226, etc. These devices may include, e.g., but not limited to, a network interface card, and modems (neither are labeled). Communications interface 224 may allow software and data to be transferred between computer system 200 and external devices. Examples of communications interface 224 may include, e.g., but may not be limited to, a modem, a network interface (such as, e.g., an Ethernet card), a communications port, a Personal Computer Memory Card International Association (PCMCIA) slot and card, etc. Software and data transferred via communications interface 224 may be in the form of signals 228 which may be electronic, electromagnetic, optical or other signals capable of being received by communications interface 224. These signals 228 may be provided to communications interface 224 via, e.g., but not limited to, a communications path 226 (e.g., but not limited to, a channel). This channel 226 may carry signals 228, which may include, e.g., but not limited to, propagated signals, and may be implemented using, e.g., but not limited to, wire or cable, fiber optics, a telephone line, a cellular link, an radio frequency (RF) link and other communications channels, etc.

In this document, the terms “computer program medium” and “computer readable medium” may be used to generally refer to media such as, e.g., but not limited to removable storage drive 214, a hard disk installed in hard disk drive and/or other storage device 212, removable storage units 218, 222, and signals, etc. These computer program products may provide software to computer system 200. The invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments of the present invention may include apparatuses and/or devices for performing the operations herein. An apparatus may be specially constructed for the desired purposes, or it may comprise a general purpose device selectively activated or reconfigured by a program stored in the device.

Embodiments of the invention may be implemented in one or a combination of hardware, firmware, and software. Embodiments of the invention may also be implemented as instructions stored on a machine-readable medium, which may be read and executed by a computing platform to perform the operations described herein. A machine-readable medium may include any mechanism for storing or transmitting information in a form readable by a machine (e.g., a computer). For example, an exemplary machine-readable storage medium may include, e.g., but not limited to, read only memory (ROM); random access memory (RAM); magnetic disk storage media; optical storage media; magneto-optical storage media; flash memory devices; other exemplary storage devices capable of storing electrical, optical, acoustical or other form of propagated signals (e.g., carrier waves, infrared signals, digital signals, etc.) thereon, and others.

Computer programs (also called computer control logic), may include object oriented computer programs, and may be stored in main memory 208 and/or the secondary memory 210 and/or removable storage units 214, also called computer program products. Such computer programs, when executed, may enable the computer system 200 to perform the features of the present invention as discussed herein. In particular, the computer programs, when executed, may enable the processor or processors 204 to provide a method to control and/or manage operation of a positioning effect detection device according to an exemplary embodiment of the present invention. Accordingly, such computer programs may represent controllers of the computer system 200.

In another exemplary embodiment, the invention may be directed to a computer program product comprising a computer readable medium having control logic (computer software) stored therein. The control logic, when executed by the processor 204, may cause the processor 204 to perform the functions of the invention as described herein. In another exemplary embodiment where the invention may be implemented using software, the software may be stored in a computer program product and loaded into computer system 200 using, e.g., but not limited to, removable storage drive 214, hard drive 212 or communications interface 224, etc. The control logic (software), when executed by the processor 204, may cause the processor 204 to perform the functions of the invention as described herein. The computer software may run as a standalone software application program running atop an operating system, or may be integrated into the operating system.

In yet another embodiment, the invention may be implemented primarily in hardware using, for example, but not limited to, hardware components such as application specific integrated circuits (ASICs), or one or more state machines, etc. Implementation of the hardware state machine so as to perform the functions described herein will be apparent to persons skilled in the relevant art(s).

In another exemplary embodiment, the invention may be implemented primarily in firmware. In yet another exemplary embodiment, the invention may be implemented using a combination of any of, e.g., but not limited to, hardware, firmware, and software, etc.

Exemplary embodiments of the invention may also be implemented as instructions stored on a machine-readable or accessible storage medium, which may be read and executed by a computing platform to perform the operations described herein. A machine-readable storage medium may include any mechanism for storing or transmitting information in a form readable by a machine (e.g., a computer). For example, a machine-readable medium may include, e.g., but not limited to, read only memory (ROM); random access memory (RAM); magnetic disk storage media; optical storage media; magneto-optical storage media; flash memory devices; other exemplary storage devices capable of storing electrical, optical, acoustical or other form of propagated signals (e.g., carrier waves, infrared signals, digital signals, etc.) thereon, and others.

The exemplary embodiment of the present invention makes reference to, e.g., but not limited to, communications links, wired, and/or wireless networks. Wired networks may include any of a wide variety of well-known means for coupling voice and data communications devices together. Alternatively, various exemplary wireless network technologies may be used to implement the embodiments of the present invention.

Unless specifically stated otherwise, as apparent from the following discussions, it may be appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

According to an exemplary embodiment, exemplary methods set forth herein may be performed by an exemplary one or more computer processor(s) adapted to process program logic, which may be embodied on an exemplary computer accessible storage medium, which when such program logic is executed on the exemplary one or more processor(s) may perform such exemplary steps as set forth in the exemplary methods.

1. A method for identifying and eliminating signals having non-physiological artifact noise from an averaged evoked potential signal in a patient, the method comprising:
recording a plurality of resultant waveforms generated by a nervous system of the patient, resulting from stimulation of one or more nerves of the nervous system;
detecting whether a non-physiological artifact noise is present in at least one resultant waveform by identifying in the at least one resultant waveform at least one characteristic representative of a signal having the non-physiological artifact noise;
classifying the at least one resultant waveform as an artifact signal if the non-physiological artifact noise is present;
removing the artifact signal from the plurality of resultant waveforms;
generating an averaged evoked potential signal from the plurality of resultant waveforms; and
generating positioning effect information about the patient using the averaged evoked potential signal.

2. The method of claim 1, further comprising stimulating the one or more nerves of the nervous system.

3. The method of claim 2, wherein the one or more nerves includes a peripheral nerve.

4. The method of claim 1, wherein the non-physiological artifact noise is pacing artifact from a pacemaker.

5. The method of claim 1, wherein the at least one characteristic is at least one from the group consisting of: a minimum slope, minimum amplitude, a maximum rise time, a maximum fall time, a minimum peak duration, and a maximum activity between a rising edge and a falling edge of the signal.

6. The method of claim 1, further comprising identifying a plurality of characteristics representative of non-physiological artifact noise by analyzing the signal for a first characteristic and if the first characteristic is present, continuing to analyze the signal for a second characteristic.

7. The method of claim 1, further comprising transmitting the positioning effect information to display or generate an alert based on the positioning effect information.

8. The method of claim 2, wherein the stimulating is achieved by at least one stimulating electrode coupled to arms and/or legs of the patient.

9. The method of claim 1, wherein the recording is achieved by at least one recording electrode coupled to a head, neck, spine, arms, legs, trunk, Erb's point and/or torso of the patient.

10. The method of claim 1, wherein the plurality of resultant waveforms used to generate the averaged evoked potential signal comprises the remaining resultant electrical waveforms of the plurality of resultant waveforms in which the artifact signal is not present.

11. An apparatus for monitoring, detecting, identifying, and eliminating non-physiological artifact noise in a physiological system from which the evoked potentials or ensemble averages are obtained, wherein the apparatus is configured to implement the method of claim 1 so as to identify artifacts in the plurality of resultant waveforms and reject those resultant waveforms that contain such artifacts from the averaged evoked potential signal.

12. The apparatus of claim 11, wherein the apparatus further comprises a system that processes electrophysiological responses sequentially looking for a series of characteristics.

13. The apparatus of claim 11, wherein the apparatus further comprises a system that processes electrophysiological responses sequentially looking for a single primary characteristic.

14. The apparatus of claim 11, wherein the apparatus further comprises a system that iteratively processes electrophysiological responses meeting a preceding identified characteristic for a further characteristic, stops processing if a characteristic is not found, and rejects an electrophysiological response from an averaged evoked potential signal if all characteristics are found.

15. The apparatus of claim 12, wherein the series of characteristics comprises but is not limited to amplitude, rise time, fall time, peak duration, and pre- and post-peak slope.

16. The apparatus in claim 11, wherein the apparatus is configured to feed information to other devices in a surgical environment that allows the other devices to identify the presence of pacemaker activity.
